(19) United States
(12) Patent Application Publication
(10) Pub. No.: US 2010/0240988 A1
(43) Pub. Date: Sep. 23, 2010

(54) Computer-aided system for 360 degree heads up display of safety/mission critical data

(76) Inventors: Kenneth Varga, Peoria, AZ (US); Joel Young, Glendale, AZ (US); Patty Cove, Glendale, AZ (US); John Hiett, Tempe, AZ (US)

Correspondence Address: Charles R. Sutton, P.O. Box 28044, Prescott Valley, AZ, 86312, US

(21) Appl. No.: 12/383,112

(22) Filed: Mar. 19, 2009

(51) Int. Cl. A61B 5/05; G09G 5/00

(52) U.S. Cl. 600/425; 345/8

(57) ABSTRACT

A safety critical, time sensitive data system for projecting safety/mission critical data onto a display pair of Commercial Off The Shelf (COTS) light weight projection glasses or monocular creating a virtual 360° HUD (Heads Up Display) with 6 degrees of freedom movement. The system includes the display, the workstation, the application software, and inputs containing the safety/mission critical information (Current User Position, Total Collision Avoidance System—TCAS, Global Positioning System—GPS, Magnetic Resonance Imaging—MRI Images, CAT scan images, Weather data, Military troop data, real-time space type markings etc.). The workstation software processes the incoming safety/mission critical data and converts it into a three dimensional space for the user to view. Selecting any of the images may display available information about the selected item or may enhance the image. Predicted position vectors may be displayed as well as 3D terrain.

### FIELD OF THE INVENTION

This invention is based primarily in the aviation field but also has applications in the medical, military, police, fire, leisure, and automotive fields as well as applications in areas requiring displaying various data onto a 3 dimensional orthogonal space. The user, simply by moving the user's head and/or eyes, achieves different views of the data corresponding to the direction of the user's gaze.

### BACKGROUND OF THE INVENTION

There are many critical perceptual limitations to humans piloting aircraft or other vehicles as well as doctors and medical technicians implementing procedures on patients, or operators trying to construct or repair equipment or structures, or emergency personnel attempting to rescue people or alleviate a dangerous situation. To overcome many of these perceptual limitations, a technique called augmented reality has been developed, to provide necessary and relevant information outside the immediate local perception of the user that is used to optimize the abilities of the user well beyond their natural local perception.

With the advent of advanced simulation technology, the augmentation of three-dimensional surfaces onto a see-through display has become more and more feasible, combined with the ability to track the orientation of an operators head and eyes and of objects in a system, or utilize known orientations of mounted see-through displays and data from sensors indicating the states of objects. The knowledge base of three-dimensional surfaces can be given the added benefit of augmentation as well as providing the ability to reasonably predict relative probabilities of collisions enabling a user to optimize the user's efforts. Such capabilities allows a user to not only have the visible world augmented, but also in conditions where the visibility is poor due to weather, night, or occlusion by structures can allow the user to have an augmented telepresence as well as a physical presence.

For pilots of aircraft, many of these limitations include occlusion by aircraft structures that keep the pilot from seeing weather conditions, icing on wings and control structures, conditions of aircraft structures, terrain, buildings, or lack of adequate day-light, as well as not knowing the flight plan, position, speed, and direction of other known aircraft, or the position, speed, and direction of unknown aircraft, structures, or flocks of birds received from radar or other sensor data.

To help overcome some of the issues of pilot occlusion, terrain data, as described in U.S. Pat. No. 4,024,539 is taught to be displayed to follow a flight plan path but does not include using head/eye orientation tracking sensors to control what is being displayed.

Obstacle avoidance is taught, in U.S. Pat. No. 5,465,142, where pilot displays are augmented by radar and laser returns, however it is limited to sensory data provided by the aircraft itself, instead of from systems outside the aircraft.

To overcome some of these limitations, U.S. Pat. No. 5,566,073 by Margolin, teaches a head mounted display system that allows a pilot to see a polygon generated terrain and human made structures superimposed as polygons on a head mounted semi-transparent display that tracks the orientation of the pilots head and allows viewing of such terrain oriented with the position of the pilots head even in directions occluded (blocked) by the aircraft structure. Margolin also discusses giving the pilot the ability to view the status of aircraft structures and functions such as by integrating fuel sensors directly with the display and pilots head orientation. Margolin discusses using aircraft radio to report identification and position of other aircraft, but does not discuss transferring flight plan or other information, such as from other aircraft out of direct radio range, as well as receiving ground radar data from other unidentified objects in the air, such as a flock of birds, or from weather data, or from other sources. Margolin also does not discuss how a heads up display could verify the normal function vs. what the actual function is of different system parts that would assist the pilot in verifying if a control surface is operating safely, obstructed, or jammed, or if it is functioning normally. Missing in the Margolin patent is also the usage of head/eye orientation tracking to control a gimbaled zoom camera to display augmented video onto a HUD display in the direction of the user's gaze or in a direction selected by the user.

Vehicle tracking information is shared between vehicles as described in both U.S. Pat. No. 5,983,161 and in U.S. Pat. No. 6,405,132 but there is no discussion of a head mounted display that tracks the position of the user's head and displays the information in direct relation to the actual direction of the objects.

For doctors and medical technicians, occlusions can be caused by static or dynamic structures of the body that occlude the operating zone of the body, or by existing equipment used with the procedure on the patient.

Further, technicians or operators that maintain vehicles or other systems have their visual perception obstructed by structures and objects that prevent them from seeing the objects and structures that need to be modified.

Eye-tracking display control, such as described in U.S. Pat. No. 6,603,491 and U.S. Pat. No. 6,847,336 can be used to control the display and keep the operator's hands free to do the work, but this prior art does not describe the use of head position and orientation tracking sensors to be used in addition to eye gaze direction for displaying an augmented reality.

Emergency personnel who require quick and safe extraction of people from a car or structure are frequently occluded by existing or damaged structure and need more optimal tactics, such as path ways that will have minimal damage to a person and optimal ease of extraction, to safely remove and rescue individuals.

Police and military personnel may have their perception occluded from building and terrain structures, as well as from weather conditions, and are missing the perception of others helping out in an operation.

The field of this invention is not limited to users of aircraft and can just as easily be applied to automobiles or vessels/vehicles of any kind such as ships, spacecraft, and submarines.

### SUMMARY OF THE INVENTION

This invention relates to displaying safety/mission critical data in real time to the user in a 3 dimensional orthogonal space to create a virtual 360° Heads Up Display (HUD). The data inputs are manipulated by a computer program (hereinafter referred to as HUD360) and displayed on either a pair of transparent Commercial Off-the-Shelf (COTS) glasses or monocle or a set of opaque COTS glasses or monocle. The glasses can be either a projection type or embedded into the display such as a flexible Organic Light Emitting Diode (OLED) display or other technology. The invention is not limited to wearable glasses, where other methods such as fixed HUD devices as well as see-through capable based hand-held displays can also be utilized if incorporated with remote head and eye tracking technologies as described in U.S. Pat. No. 6,603,491 and U.S. Pat. No. 6,847,336 or by having orientation sensors on the device itself.

The pilot can use the HUD360 display to view terrain, structures, and other aircraft nearby and other aircraft that have their flight plan paths in the pilot's vicinity as well as display this information in directions that are normally occluded by aircraft structures or poor visibility.

Aside from viewing external information, the health of the aircraft can also be checked by the HUD360 by having a pilot observe an augmented view of the operation or structure of the aircraft, such as of the aileron control surfaces, and be able to see an augmentation of set, min, or max, control surface position. The actual position or shape can be compared with an augmented view of proper (designed) position or shape in order to verify safe performance, such as degree of icing, in advance of critical flight phases, where normal operation is critical such as during landing or take off. This allows a pilot to be more able to adapt in abnormal circumstances where operating surfaces are not functioning optimally.

Pan, tilt, and zoom cameras mounted in specific locations to see the outside of the aircraft can be used to augment the occluded view of the pilot, where said cameras can follow the direction of the pilots head and allow the pilot to see the outside of what would normally be blocked by the flight deck and vessel structures. For instance, an external gimbaled infrared camera can be used for a pilot to verify the de-icing function of aircraft wings to help verify that the control surfaces have been heated enough by verifying a uniform infrared signature and comparing it to expected normal augmented images. A detailed database on the design and structure, as well as full motion of all parts can be used to augment normal operation that a pilot can see, such as minimum maximum position of control structures. These minimum maximum positions can be augmented in the pilots HUD so the pilot can verify control structures' operation whether they are dysfunctional or operating normally.

In another example, external cameras in both visible and infrared spectrum on a space craft can be used to help a astronaut easily and naturally verify the structural integrity of the spacecraft control surfaces, that may have been damaged during launch, or to verify the ability of the rocket boosters to contain plasma thrust forces before and during launching or re-entry to earths atmosphere and to determine if repairs are needed and if an immediate abort is needed.

With the use of both head and eye orientation tracking, objects normally occluded in the direction of a user's gaze (as determined both by head and eye orientation) can be used to display objects hidden from normal view. This sensing of both the head and eye orientation can give the user optimal control of the display augmentation as well as an un-occluded omnidirectional viewing capability freeing the user's hands to do the work necessary to get a job done simultaneously and efficiently.

The user can look in a direction of an object and either by activating a control button or by speech recognition selects the object. This can cause the object to be highlighted and the system can then provide further information on the selected object. The user can also remove or add layers of occlusions by selecting and requesting a layer to be removed. As an example, if a pilot is looking at an aircraft wing, and the pilot wants to look at what is behind the wing, the pilot can select a function to turn off wing occlusion and have video feed of a gimbaled zoom camera positioned so that the wing does not occlude it. The camera can be oriented to the direction of the pilots head and eye gaze, whereby a live video slice from the gimbaled zoom camera is fed back and projected onto the semi transparent display onto the pilot's perception of the wing surface as viewed through the display by perceptual transformation of the video and the pilots gaze vector. This augments the view behind the wing.

The pilot or first officer can also select zoom even further behind the wing surface or other structure, giving beyond the capability of an “eagle eye” view of the world through augmentation of reality and sensor data from other sources, where the user's eyes can be used to control the gimbaled motion of the zoomable telescopic camera.

As another application to aid the captain or first officer in security detail of the flight deck, the captain or first officer can turn their head looking back into the cabin behind the locked flight deck door and view crew and passengers through a gimbaled zoom camera tied into the captain's or first officer's head/eye orientations to assess security or other emergency issues inside the cabin or even inside the luggage areas. Cameras underneath the aircraft can also be put to use by the captain or first officer to visually inspect the landing gear status, or check for runway debris well in advance of landing or takeoff, by doing a telescopic scan of the runway.

Gimbaled zoom camera perceptions, as well as augmented data perceptions (such as known 3D surface data, 3D floor plan, or data from other sensors from other sources) can be transferred between pilot, crew, or other cooperatives with each wearing a gimbaled camera (or having other data to augment) and by trading and transferring display information. For instance, a first on the scene fire-fighter or paramedic can have a zoom-able gimbaled camera that can be transmitted to other cooperatives such as a fire chief, captain, or emergency coordinator heading to the scene to assist in an operation. The control of the zoom-able gimbaled camera can be transferred allowing remote collaborators to have a telepresence (transferred remote perspective) to inspect different aspects of a remote perception, allowing them to more optimally assess, cooperate and respond to a situation quickly.

### BRIEF DESCRIPTION OF THE FIGURES

The COTS glasses can contain a 6-degree of freedom motion sensor, eye tracking sensors, and compass sensor. The COTS glasses may also be connected using a physical cable connection or may be connected by a wireless technology such as Wireless Fidelity (WiFi). This invention can be more fully understood from the following detailed description when taken in conjunction with the accompanying drawings, in which:

FIG. 1A is a HUD360 system block diagram of a pair of projection type COTS glasses showing a microphone, earphones, and sensors with eye and head tracking;

FIG. 1B is a high-level system block diagram of multiple HUD360's.

FIG. 2 is a diagram of a pair of projection type COTS glasses with optional microphone and earphones shown;

FIG. 3A is an augmented pilot view with aircraft flight plan view with critical and caution terrain shown, along with a “Traffic out of sight” indicator;

FIG. 3B is an augmented pilot view with aircraft flight plan view with critical and caution terrain shown

FIG. 3C is an augmented pilot view with aircraft flight plan view with caution terrain shown

FIG. 4A is an augmented pilot view with aircraft flight plan ribbon displayed with non-critical terrain;

FIG. 4B is an augmented pilot view with aircraft flight plan ribbon displayed with a collision course warning with another aircraft above non-critical terrain;

FIG. 5 is an augmented pilot view of both terrain and of ground structures, where structures that are dangerous to the flight plan path are highlighted in the display.

FIG. 6 shows a hand-held pointing device that is used for controlling a display;

FIG. 7 shows Air Traffic Control (ATC) tower view without aircraft flight plan and ATC entered flight procedures;

FIG. 8 shows ATC tower view with flight data;

FIG. 9 shows ATC tower view with flight data and air collision alert;

FIG. 10 shows ATC tower view with flight data and ground collision alert;

FIG. 11 shows ATC tower view with lost signal and coasting;

FIG. 12 ATC Regional Control Center (RCC) view;

FIG. 13 is an augmented pilot view with predicted position vector shown with no other outside aircraft data.

FIG. 14 ATC/RCC pilot's view from aircraft perspective;

FIG. 15 military battlefield view—Map view;

FIG. 16 military battlefield view—Map view Army Operations;

FIG. 17 military battlefield view—Map view Naval Operations;

FIG. 18 military battlefield view—Augmented Ground view;

FIG. 19 military Control Center (MCC) view from battlefield perspective;

FIG. 20 ATC Tower view with weather;

FIG. 21 pilot view with weather;

FIG. 22 battlefield view with weather;

FIG. 23 shows a HUD360 application for navigating on a river, bay, or ocean with distance to object displayed;

FIG. 24 shows a HUD360 application optimizing a search and rescue operation with a team of coast guard vessels optimized coordination of search areas with current flows identifying explored and unexplored areas;

FIG. 25 shows a HUD360 application for a team of search and rescue units on a mountain displaying explored and unexplored areas;

FIG. 26 shows a HUD360 application for a team of firefighters, police, or swat team in a multi-story building;

FIG. 27 shows a HUD360 application for emergency vehicles to optimize routing through traffic;

FIG. 28 shows a HUD360 application for leisure hikers;

FIG. 29 shows a HUD360 application for a police/swat hostage rescue operation;

FIG. 30 shows a HUD360 application for leisure scuba divers;

FIG. 31 shows a HUD360 application for real estate agents on the street;

### DETAILED DESCRIPTION

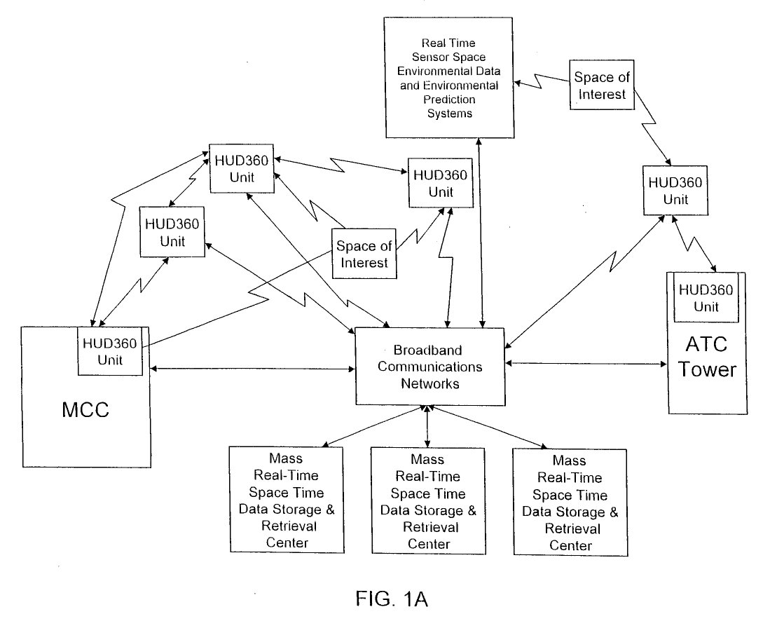

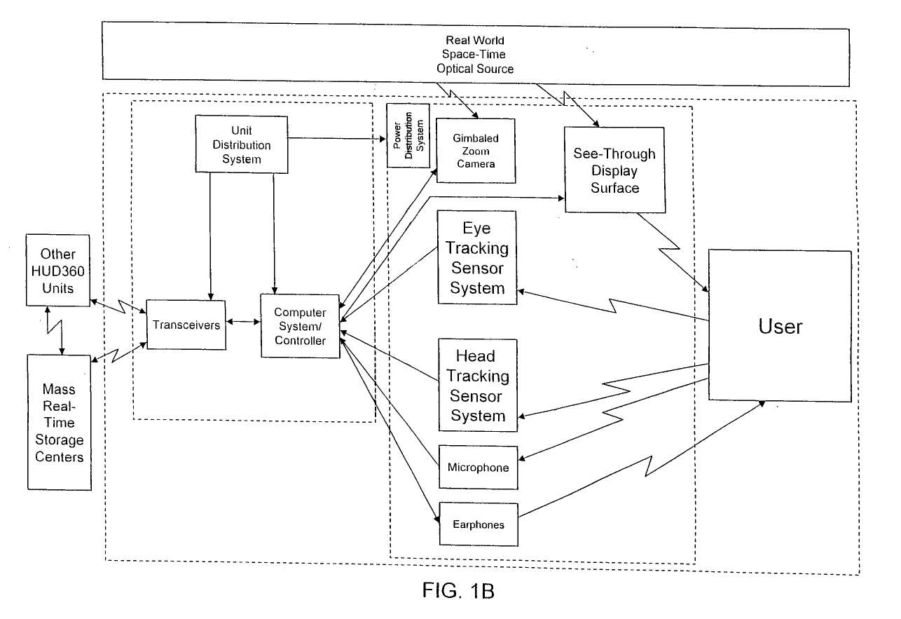

A functional system block diagram of a HUD360 1 system with see-through display surface 4 viewed by a user 6 of a space of interest 112 is shown in FIG. 1A. In some applications, the HUD360 1 see-through display surface 4 can be set in an opaque mode where the entire display surface 4 has only augmented display data where no external light is allowed to propagate through display surface 4. Other features of the HUD360 1 system include a head tracking sub-system 110, an eye tracking sub-system 108, and a microphone 5 are all shown in FIG. 1A and all of which can be used as inputs with the ability to simultaneously control the augmented see-through display view 4, or to control another available system of the user's 6 choice. Also shown is a pair of optional earphones 11 which can also be speakers to provide output to user 6 that can complement the augmented output of the see-through display surface 4. Also shown in FIG. 1A is an optional gimbaled zoom camera that can be a lone camera or multiple independent cameras of various types that the user 6 or outside user(s) 6 of the system can view and control in real-time. The camera(s) 106 can be mounted on the goggles as an embedded part of the HUD360 1 system as shown in FIG. 1A, or elsewhere and integrated as appropriate. Sensing and communications between user 6 and see-through display 4 eye tracking sensor system 108, head tracking sensor system 110, microphone 5, earphones 11, and hand-held pointing device 24 are shown as wireless, while to real-time computer system/controller 102 they are shown as wired directly but can be wireless or wired depending on the desired application. All the functional blocks shown within HUD360 1 can be embedded or mounted within the goggles, worn by the user, or can be fixed away from the user 6 depending on the desired application.

Real-time computer system/controller 102 is shown in FIG. 1A to primarily augment see-through display 4, route and/or process signals between the user 6, camera (s) 106, eye-tracking sensor system 108, head tracking sensor system 110, microphone 5, earphones/speakers 11, hand held pointing (or other input such as a wireless keyboard and/or mouse) device 24 and transceiver 100 to other HUD360 1 units directly, or to other broadband communications networks 25. Power distribution system 104 can be controlled by real-time computer system/controller 102 to optimize portable power utilization, where the power is distributed to all the functional blocks of the HUD360 1 unit that are mobile needing power and turned on, off, or low power state as needed to minimize power losses. Transceiver 100 can also serve as a repeater, router, or bridge to efficiently route broadband signals from other HUD360 1 devices as a contributing part of a distributed broadband communications network 25 shown in FIG. 1B. Transceiver 100 can be made to send receive data such as Automatic Dependent Surveillance—Broadcast (ADS-B) data, but transceiver 100 is not limited to ADS-B, or to radio technology and can include other forms of transmission media such as from optical laser technology that carries traffic data or other collected data from other HUD360 1 units directly, indirectly, or receive data from mass real-time space data storage & retrieval centers 114 shown in FIG. 1B.

FIG. 1B is a high-level system view of multiple HUD360's 1 cooperating together independently, or as part of an Air Traffic Control (ATC) Tower 27, or Military Control Center (MCC) 12 or other control center, not shown. The HUD360 1 units are shown to utilize direct path communications between each other if within range, or by using broadband communications networks 25 that can include terrestrial (ground networks) or extra-terrestrial (satellite) communication systems. The HUD360 1 unit can share information about spaces of interest 112 by communicating directly with each other, or through broadband communications networks 25. In addition, the HUD360 1 units can read and write to real-time space data storage & retrieval centers 114 via the broadband communications networks 25. Predicted data can also be provided by real-time sensor space environmental prediction systems 46 such as from radars or satellite. All systems and data can be synchronized and standardized to common or multiple atomic clocks, not shown, and weighted accordingly by time reliability and probabilities, to improve accuracy and precision of real-time data.

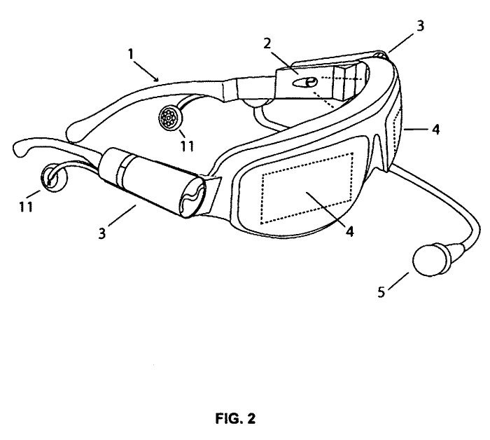

Shown in FIG. 2 is a preferred lightweight COTS HUD360 1 see-through goggles with display projection source that can also contain optional eye-tracking sensors 2, head orientation sensors 3, see-through display surfaces in the user's view 4, optional microphone 5, and optional earphones 11. The display surface 4 is primarily used to augment the optical signals from the environment (space of interest 112 not shown) outside with pertinent data useful to the user of the display. This augmented data can be anything from real-time information from sensors (such as radars, cameras, real-time databases, satellite, etc.), or can implement applications used on a typical desk top computer laptop, cell phone, or hand held device such as a Personal Digital Assistant (PDA) where internet web browsing, text messages, e-mail, can be read from a display or through text to speech conversion to earphones 11 or written either by manually entering using an input device such as the eyes to select letters, or by an external input device such as a keyboard or mouse wirelessly integrated with HUD360 1, or by speech to text conversion by user speaking into microphone 5 to control applications.

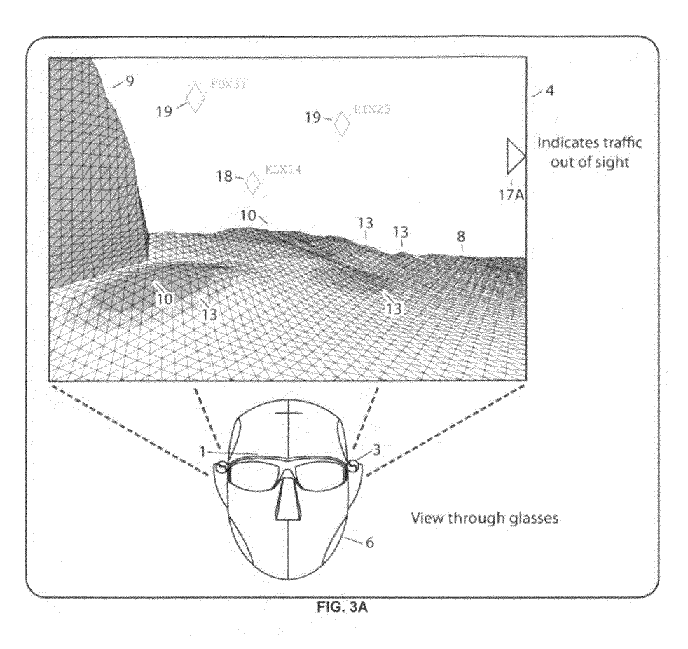

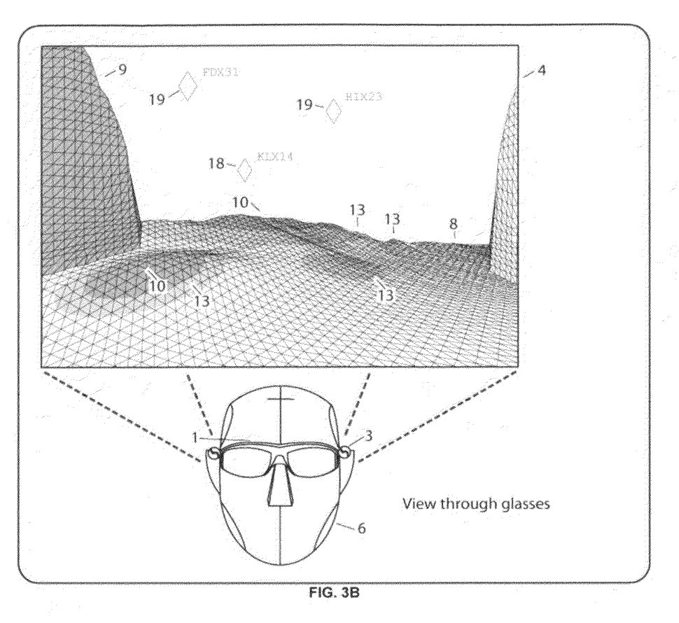

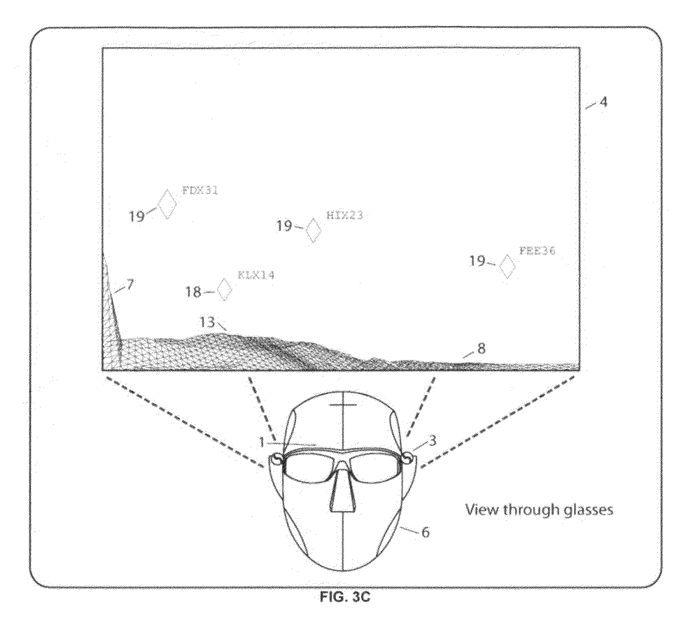

An augmented perception of a pilot view with a HUD360 1 is shown in FIGS. 3A, 3B, 3C, 4A, 4B, 5, 13 and FIG. 21.

FIG. 3A shows the augmented perception of a pilot view using a HUD360 1 where safe terrain surface 8, cautionary terrain surface 13, and critical terrain surfaces 9 and 10 are identified and highlighted. Aircraft positions are also augmented on the HUD360 1 display as an aircraft 18 on a possible collision course with critical terrain surface 9 as a mountain on the left of the see through display view 4 (can be displayed in red color to differentiate, not shown in the FIG.). Also shown is aircraft 19 not on a possible collision course (can be displayed in another color not shown in the FIG., such as green, to differentiate from possible collision course aircraft 18). Aircraft out of sight 17A is augmented on the see-through display views 4 that is shown in the direction relative to the pilot's direction of orientation, are indicated in their direction on the see-through display edge and can be colored accordingly to indicate if it is an out-of-sight collision course (not shown) or non-collision course aircraft 17A. Other out of sight indicators not shown in the figure can be displayed and are not limited to aircraft such as an out-of-sight indicator for an obstruction or mountain, etc, and the seriousness of the obstruction can be appropriately indicated such as by color or flashing, etc. Aircraft out of sight and on a collision course can also be indicated in their direction to see on the display edge though not shown in the figures. Critical surface 10 can be colored red or some other highlight so that it is clear to the pilot that the surface is dangerous. Cautionary surface 13 can be colored yellow or some other highlight so that it is clear to the pilot that the surface can become a critical surface 10 if the aircraft gets closer or if the velocity of the aircraft changes such that the surface is dangerous. Safe terrain surface 8 can be colored green or some other highlight so that it is clear to the pilot that the surface is not significantly dangerous. Other highlights or colors not shown in the figures can be used to identify different types of surfaces such as viable emergency landing surfaces can also be displayed or colored to guide the pilot safely down.

Aircraft direction, position, and velocity are also used to help determine if a landscape such as a mountain or a hill is safe and as shown in FIG. 3B this terrain is highlighted as a critical surface 9 (can be colored red) or as a safe terrain surface 8 (can be colored green). These surfaces can be highlighted and/or colored in the see-through display view 4 so that it is clear to the pilot which surface needs to be avoided and which surface is not significantly dangerous to immediately fly towards if needed.

FIG. 3C shows another view through the HUD360 1 with no critical surfaces highlighted, but a cautionary surface 13, and safe terrain surface 8 along with aircraft not on collision course 19 as well as an aircraft 18 on a possible collision course. Not shown in the figures, a critical terrain (9 or 10) out of view indicator can also be displayed on the edge of the see-through display in the direction of the critical terrain out of view.

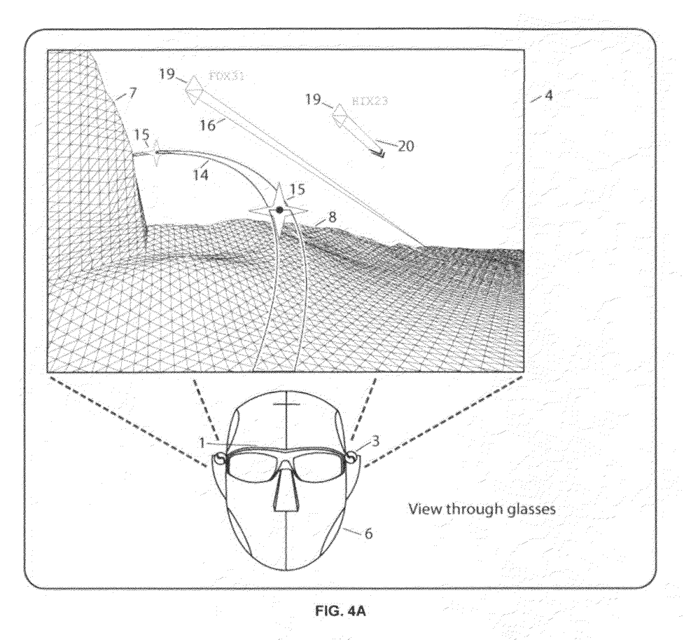

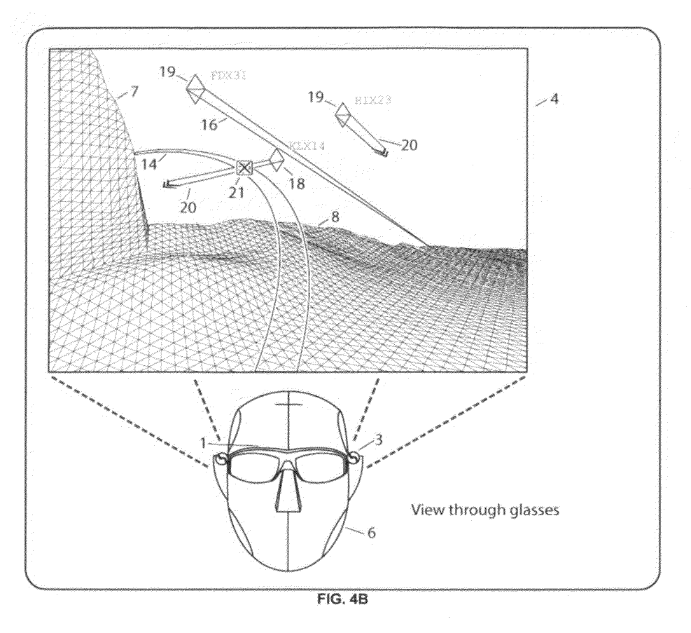

Shown in FIG. 4A is another view of the HUD360 1 with no critical surfaces highlighted, shows the pilot's aircraft flight plan path 14 with two way points identified 15, with aircraft 19 that has a known flight plan 16 displayed along with another aircraft 19 with only a predicted position vector 20 known. The predicted position vector 20 is the predicted position the pilot must respond to, in order to correct the course in time, and is computed by the velocity and direction of the vessel.

A possible collision point 21 is shown in FIG. 4B in see through display view 4 where the HUD360 1 shows the pilot's aircraft flight plan path 14 intersecting at predicted collision point 21 with aircraft 18 with known predicted position vector 20 all over safe terrain surfaces 8 and 7.

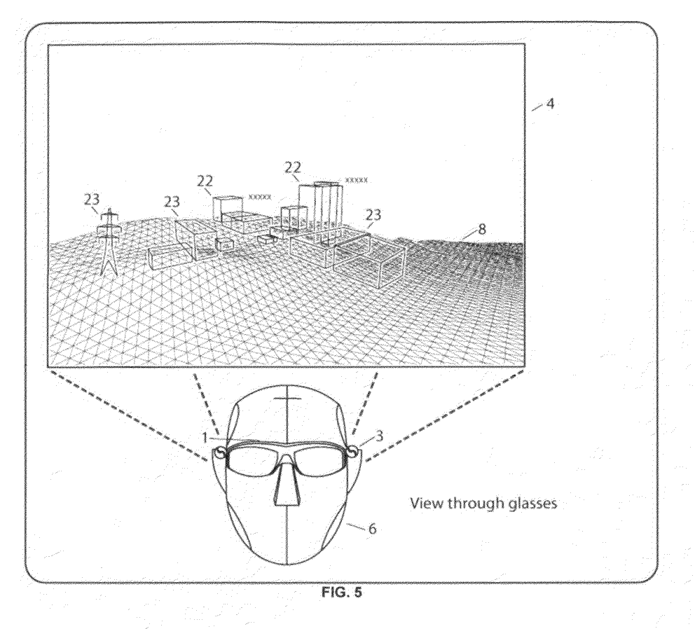

Critical ground structures 22 are highlighted in the HUD360 1 pilot view 4 in FIG. 5 where non-critical structures 23 are also shown in the see-through display view 4 on HUD360 1 on top of non-critical terrain surface 8.

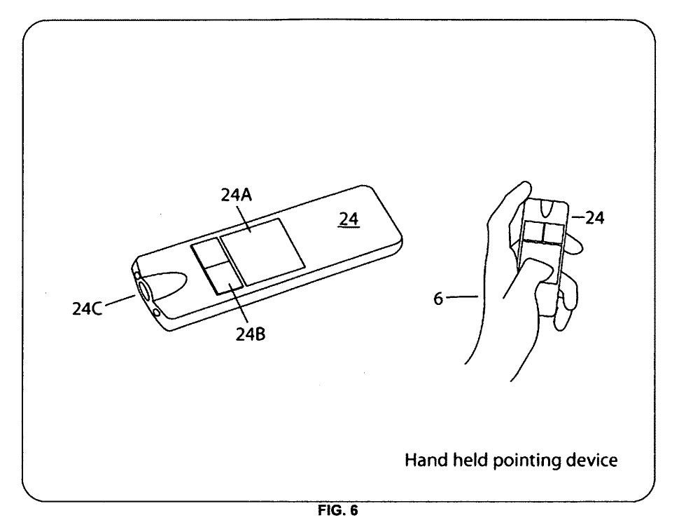

FIGS. 6, 7, 8, 9, 10, 11 and 12 show another embodiment of the invention as an augmented perspective of an air traffic controller inside an Air Traffic Control (ATC) tower.

A pointing device 24 in FIG. 6 is used by user 6 to control a Heads-Up Display (HUD) with thumb position sensor 24A, mouse buttons 24B, and pointing sensor 24C that can also serve as a laser pointer.

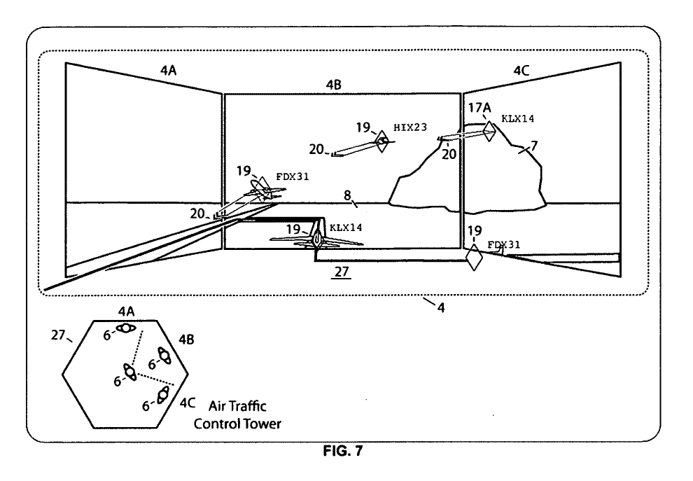

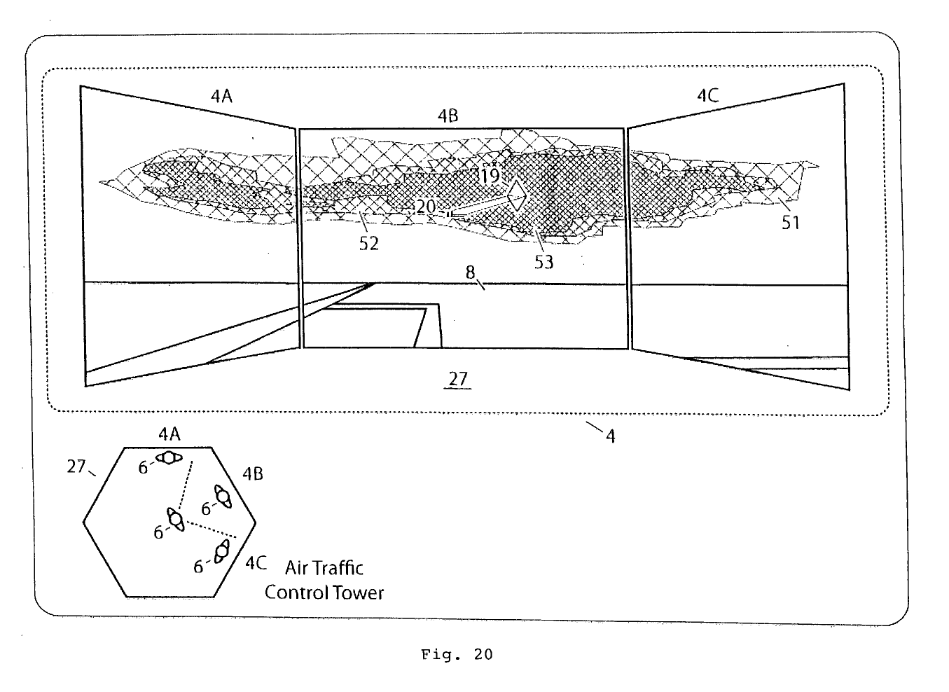

Three planar windows (4A, 4B, and 4C) with a HUD360 1 display view 4 are shown from inside an ATC tower in FIG. 7 where three aircraft 19 in window 4B with a third aircraft 19 in window 4C occluded by non-critical mountain surface 7 with predicted position vectors 20 and a forth aircraft 19 shown at bottom of window 4C. Also shown in FIG. 7 is a top view of the ATC tower with four viewing positions shown inside the tower, where 4A, 4B, and 4C are the tower windows, with the upper portion of FIG. 7 as the center perspective centered on window 4B, with window 4A and 4C also in view. Although not shown in FIG. 7 through 11, all window surfaces (Omni-directional) of the ATC tower windows can have a fixed HUD display surface 4 where the augmented view can apply, and further a see-through or opaque HUD 4 on the ceiling of the tower can also be applied as well as out of sight aircraft indicators (17A and 17B) displayed on the edge of the display nearest the out-of-sight aircraft position, or a preferred embodiment with HUD360 light weight goggles 1 can be used in place of the fixed HUD's. Safe terrain surface 8 and safe mountain surface 7 is shown in FIGS. 7 through 11 and safe terrain surface 8 is shown in FIG. 20. Although not shown in FIG. 7 through 11 and in FIG. 20, critical surfaces 9, 10, cautionary terrain surfaces 13, and critical structures 22 can be augmented and displayed to the ATC personnel to make more informative decisions on optimizing the direction and flow of traffic.

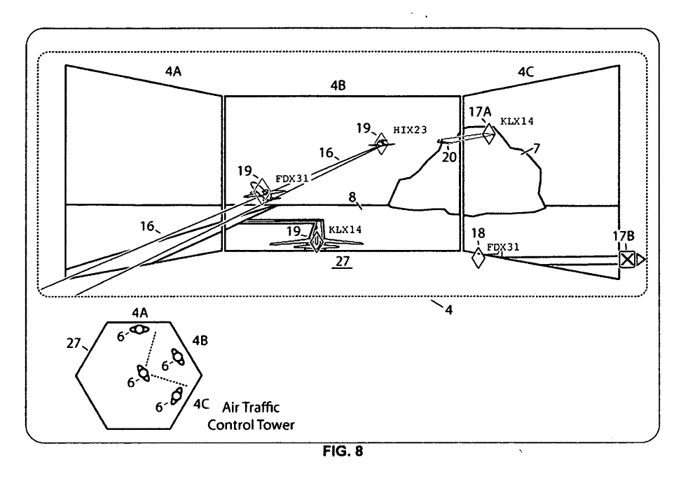

FIG. 8 shows a total of six aircraft being tracked see-through display view 4 from an ATC tower perspective. Three aircraft 19 are shown in-sight through ATC window 4B that are not on collision courses with flight plan paths 16 shown. In ATC window 4C an out of sight aircraft 17A occluded by non-critical mountain surface 7 is shown with predicted position vector 20. Also shown in FIG. 8, through window 4C, is out of sight indication 17B of a collision bound aircraft heading towards probable collision aircraft 18 augmented on bottom of window 4C.

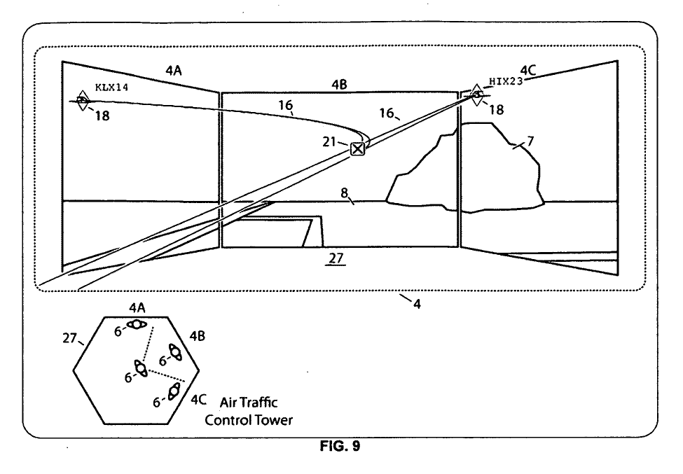

FIG. 9 shows an ATC tower 27 see-through display view 4 from a user 6 looking at ATC windows 4A, 4B, and 4C where two aircraft 18 on a predicted air collision course point 21 along flight plan paths 16 derived from flight data over safe terrain 8 and safe mountain surface 7.

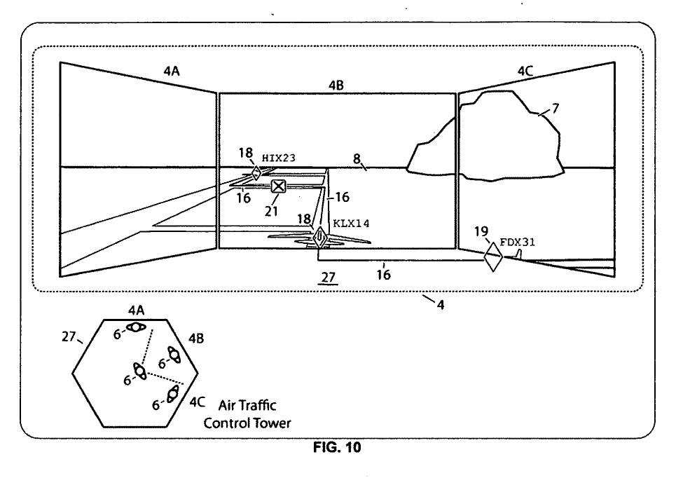

FIG. 10 shows an ATC tower 27 see-through display view 4 with a predicted ground collision point 21 between two aircraft 18 with flight plan paths 16 on safe surface 8 with safe mountain surface 7 shown. User 6 see-through display view 4 is shown from user seeing through ATC windows 4A, 4B, and 4C. Aircraft 19 that is not on a collision course is shown through ATC window 4C.

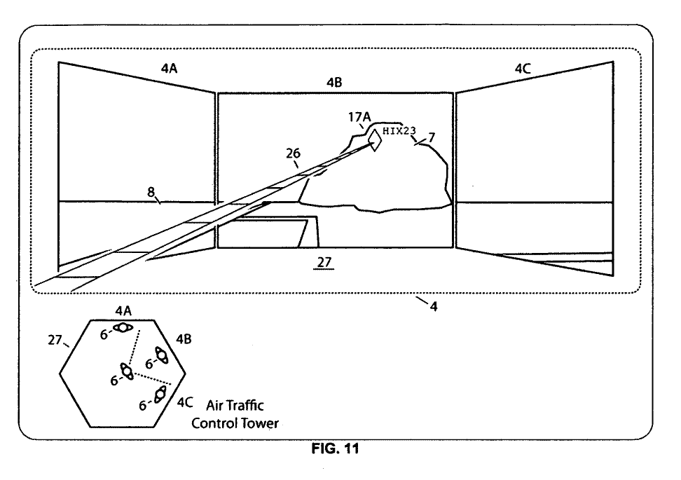

FIG. 11 shows an ATC tower 27 see-through display view 4 from user 6 seeing through ATC windows 4A, 4B, and 4C. An aircraft 17A is occluded by a determined as safe mountain terrain surface 7 from last known flight data, where the flight data is latent, with the last predicted flight plan path 26 shown over safe terrain surface 8. The safe mountain terrain surface 7 is identified as safe in this example and in other examples in this invention, because the last known position of the aircraft was far enough behind the mountain for it not to be a threat to the aircraft 17A.

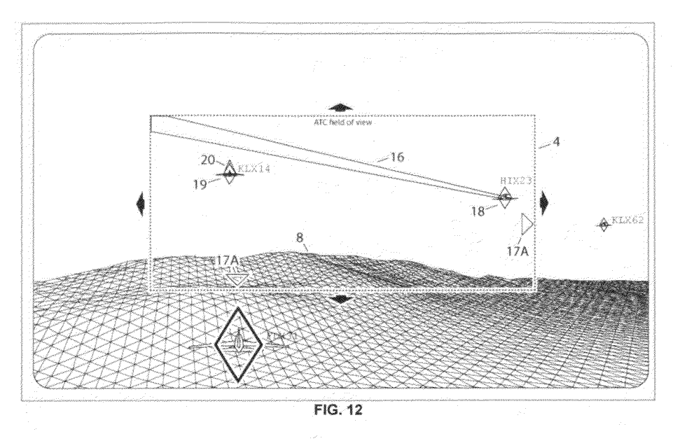

For regional ATC perspective, FIG. 12 demonstrates a telepresence view of a selected aircraft on an ATC display field of view 4 (with the ATC HUD360 1 display view 4 in opaque or remote mode) over probable safe terrain surface 8 with one aircraft 19 in sight with predicted position vector 20 shown, that is not on a collision course. A second aircraft 18 in sight and on a collision course from aircraft predicted position data is shown (with collision point 21 outside of view and not shown in FIG. 20). Out of sight aircraft indicators 17A are shown on the bottom and right sides of the ATC field of view display 4 to indicate an aircraft outside of display view 4 that are not on a collision course. The ATC regional HUD360 1 user 6 (not shown) can move the display view 4 (pan, tilt, zoom, or translate) to different regions in space to view different aircraft in real-time, such as the aircraft shown outside display view 4 and rapidly enough to advert a collision.

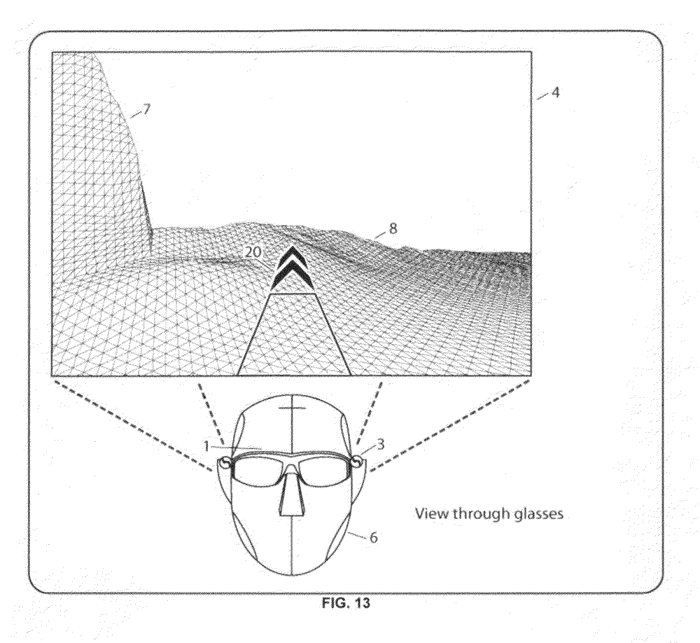

FIG. 13 shows a pilot display view 4 with predicted position vector 20 over safe terrain surface 8, but no flight plan data is displayed.

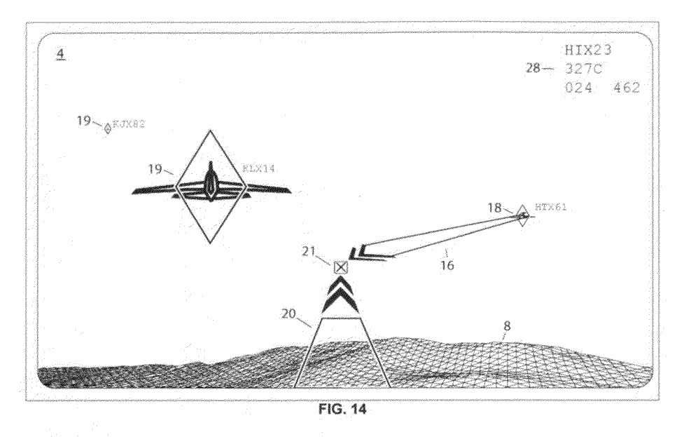

FIG. 14 provides an ATC or Regional Control Center (RCC) display view 4 of a selected aircraft identified 28 showing predicted aircraft predicted position vector 20 over safe terrain surface 8 along with two in-sight aircraft 19 that are not on a collision course, and a third in-sight aircraft 18 that is on a predicted collision point 21 course along flight plan path 16.

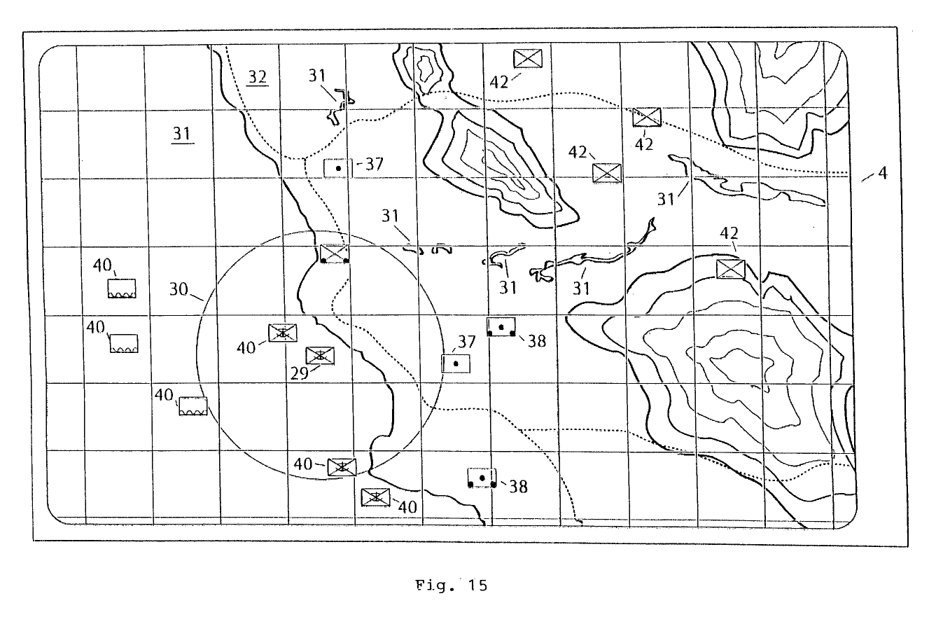

FIGS. 15, 16, 17, 18, and FIG. 19 demonstrate a display view 4 of different battlefield scenarios where users can zoom into a three dimensional region and look at and track real time battle field data, similar to a flight simulator or “Google Earth” application but emulated and augmented with real-time data displayed, as well as probable regional space status markings displayed that can indicate degree of danger such as from sniper fire or from severe weather. The system user can establish and share telepresence between other known friendly users of the system, and swap control of sub-systems such as a zoom-able gimbaled camera view on a vehicle, or a vehicle mounted gimbaled weapon system if a user is injured, thereby assisting a friendly in battle, or in a rescue operation. Users of the system can also test pathways in space in advance to minimize the probability of danger by travelling through an emulated path in view 4 accelerated in time, as desired, identifying probable safe spaces 34 and avoiding probable cautious 35 and critical 36 spaces that are between the user's starting point and the user's planned destination. A user can also re-evaluate by reviewing past paths through space by emulating a reversal of time. The identification of spaces allows the user to optimize their path decisions, and evaluate previous paths.

In FIG. 15 battlefield data of all unit types is shown on a three-dimensional topographical display view 4 in real time where a selected military unit 29 is highlighted to display pertinent data such as a maximum probable firing range space 30 over land 32 and over water 31. The probable unit maximum firing range space 30 can be automatically adjusted for known physical terrain such as mountains, canyons, hills, or by other factors depending on the type of projectile system. Unit types in FIG. 15 are shown as probable friendly naval unit 40, probable friendly air force unit 37, probable friendly army unit 38, and probable unfriendly army unit 42.

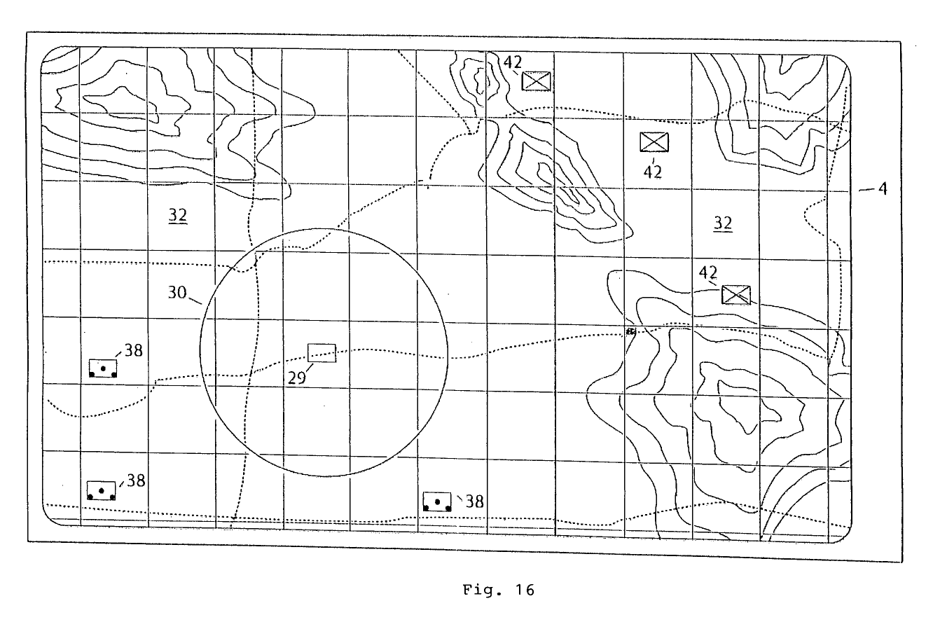

FIG. 16 shows an aerial battlefield view 4 with selected unit 29 on land 32. The selected unit 29 is identified as a probable motorized artillery or anti-aircraft unit with a probable maximum unit firing space 30 near probable friendly army units 38. Probable unfriendly army units are shown on the upper right area of FIG. 16.

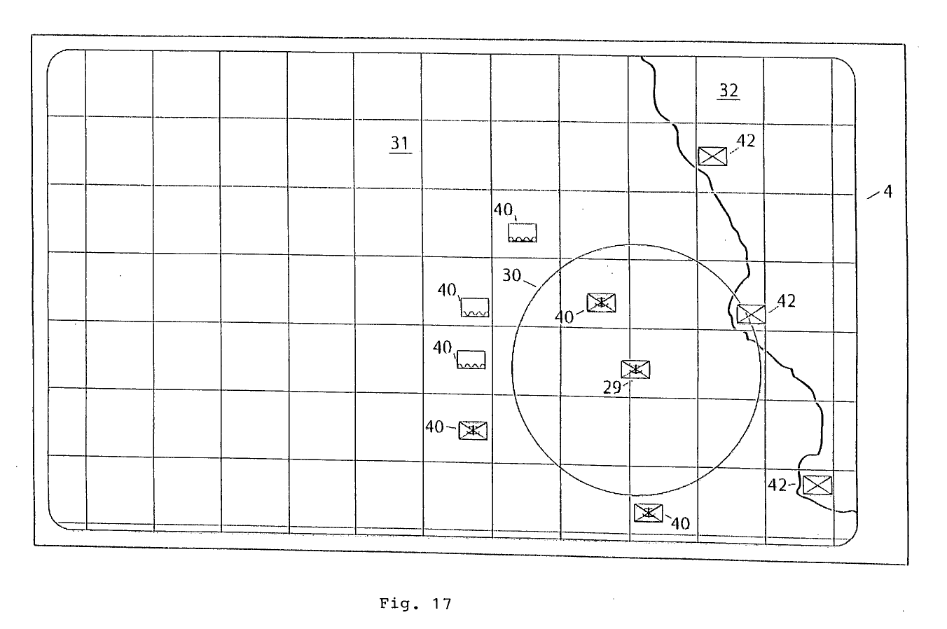

FIG. 17 shows a naval battlefield view 4 with selected unit 29 on water 31 with probable firing range 30 along with probable friendly navy units 40 along with probable unfriendly army units 42 on land 32.

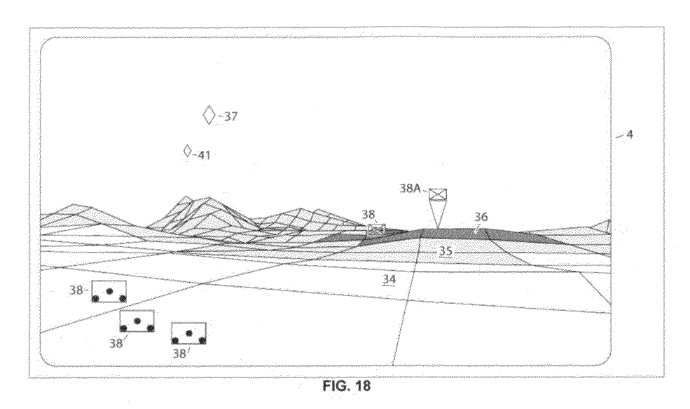

FIG. 18 shows a military battlefield view 4 with probable friendly army units 38 and out of sight probable friendly army unit 38A, and probable unfriendly air-force unit 41 being intercepted by probable friendly air-force unit 37 (evidence of engagement, although not explicitly shown in the FIG., such as a highlighted red line between probable unfriendly air-force unit 41 and probable friendly air-force unit 37, or some other highlight, can be augmented to show the engagement between units). Probable safe spaces (“green zone”) 34, probable cautious battle spaces (“warm yellow zone”) 35, and probable critical battle spaces (“red hot zone”) 36, all of which are weighted in probability by time and reporting, are also shown in FIG. 18. The battle space status types 34, 35, and 36, can be determined by neural network, fuzzy logic, known models, and other means with inputs of reported weighted parameters, sensors, and time based decaying weights (older data gets deemphasized where cyclical patterns and recent data get amplified and identified). Unit types are not limited to the types described herein but can be many other specific types or sub-types reported, such as civilian, mobile or fixed anti-aircraft units, drones, robots, and mobile or fixed missile systems, or underground bunkers. Zone space type identification can be applied to the other example applications, even though it is not shown specifically in all of the figures herein. The terrain status types are marked or highlighted on the display from known data sources, such as reports of artillery fire or visuals on enemy units to alert other personnel in the region of the perceived terrain status.

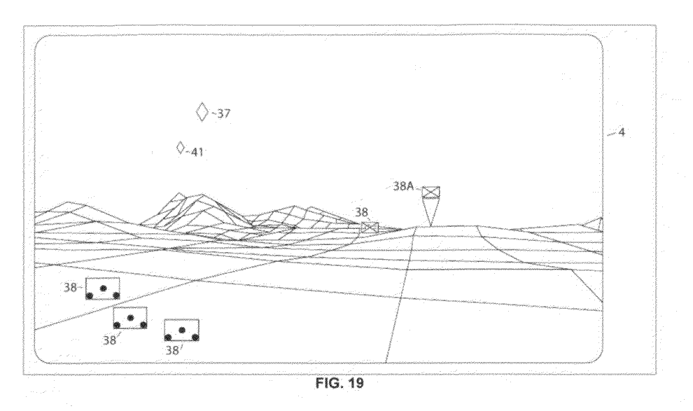

In FIG. 19 a Military Control Center (MCC) perspective view 4 of a battle space with zone spaces not shown but with probable friendly army units 38 and out of sight probable friendly army unit 38A, and probable unfriendly air-force unit 41 being intercepted by probable friendly air-force unit 37.

FIGS. 20, 21, 22, and 23 show weather spaces in ATC, pilot, ground, and marine views 4. In FIG. 20, an ATC tower 27 display view 4 with an out of sight aircraft 17A with probable predicted non-collision course predicted position vector 20 but is occluded by critical weather space 53 (extreme weather zone, such as hurricane, tornado, or typhoon) above probable safe terrain surface 8. Other weather spaces marked as probable safe weather space 51 (calm weather zone), and probable cautious weather space 52 (moderate weather zone) are all shown in FIG. 20. A top-down view of ATC tower 27 is shown on the bottom left of FIG. 20 with multiple users' 6 viewing through ATC windows 4A, 4B, 4C.

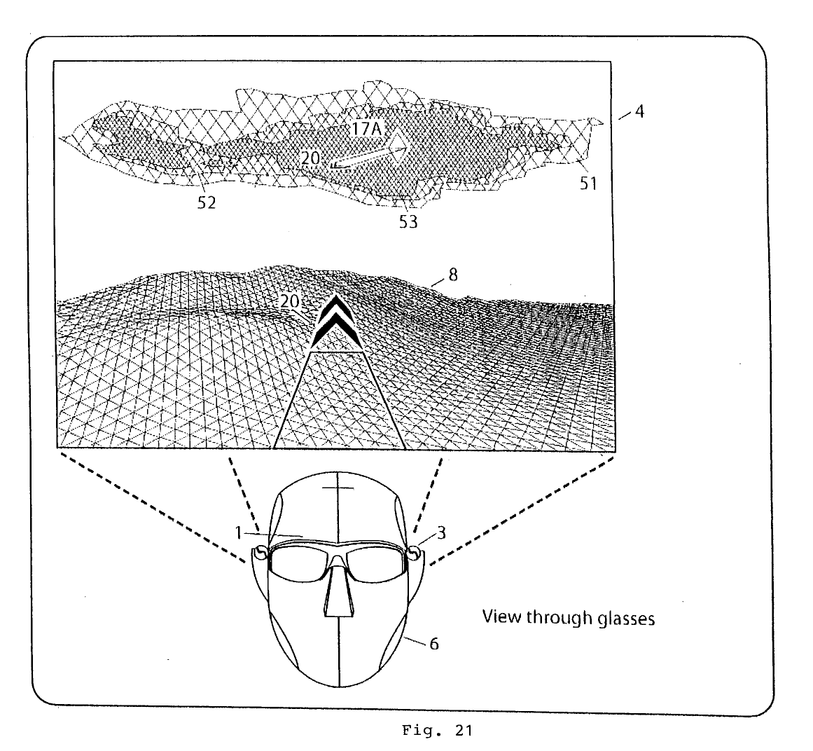

In FIG. 21 is a pilot display view 4 with an out of sight aircraft 17A not on a predicted collision course, but occluded directly behind critical weather space 53 but near probable safe weather space 51 and probable cautious weather space 52. Also shown are probable safe terrain surface 8 and pilots' probable predicted position vectors 20.

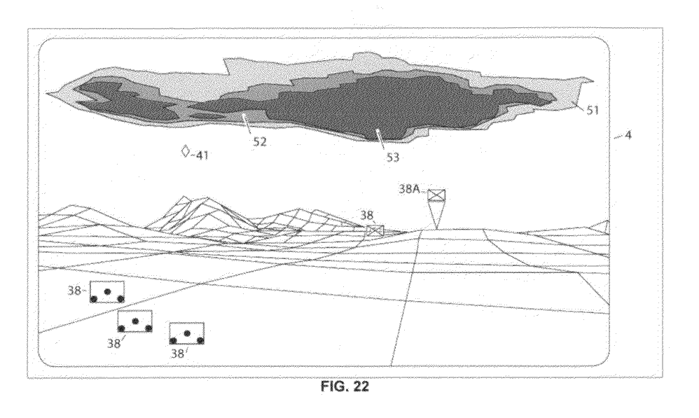

In FIG. 22 is a battle field view 4 with weather spaces marked as probable safe weather space 51, probable cautious weather space 52, and probable critical weather space 53 with probable unfriendly air force unit 41 and probable friendly in-sight army units 38. Although not shown, probable friendly and probable unfriendly units can be identified and augmented with highlights such as with different colors or shapes and behavior to clarify what type (probable friendly or probable unfriendly) it is identified as. Many techniques can be used to determine if another unit is probably friendly or probably not friendly, such as time based encoded and encrypted transponders, following of assigned paths, or other means.

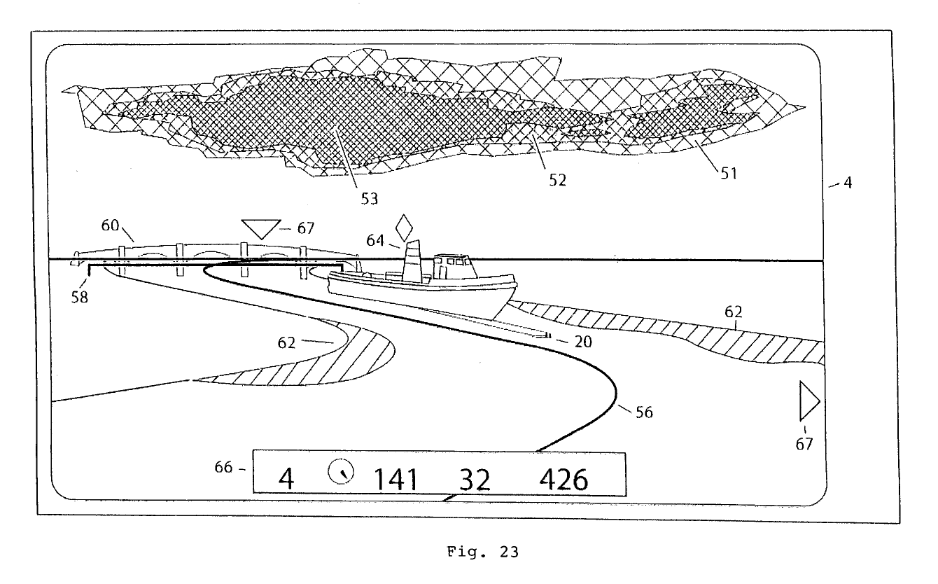

In FIG. 23 a HUD360 1 marine application is shown through display view 4 having navigation path plan 56 with approaching ship 64 with predicted position vector 20, dangerous shoals 62, essential parameter display 66, bridge 60, unsafe clearance 58, an out-of-sight ship indicator 67 behind bridge 60 and at bottom right of display view 4. Also shown are critical weather space 53, probable safe weather space 51, and probable cautious weather space 52. Not shown in FIG. 23 but display view 4 can be augmented with common National Oceanographic and Atmospheric Administration (NOAA) chart data or Coastal Pilot items such as ship wrecks, rocky shoals, ocean floor types or other chart data. This is also applicable for aviation displays using similar augmentation from aeronautical chart data. Also not shown in FIG. 23, but can be augmented is the surface and depth of the floor of the ocean, river, or channel, or lake, along with tidal, river, or ocean current vectors on the water, known probable fishing net lines, moors, wind direction and magnitude indication, navigation buoy augmentations, as well as minimum and maximum tide levels.

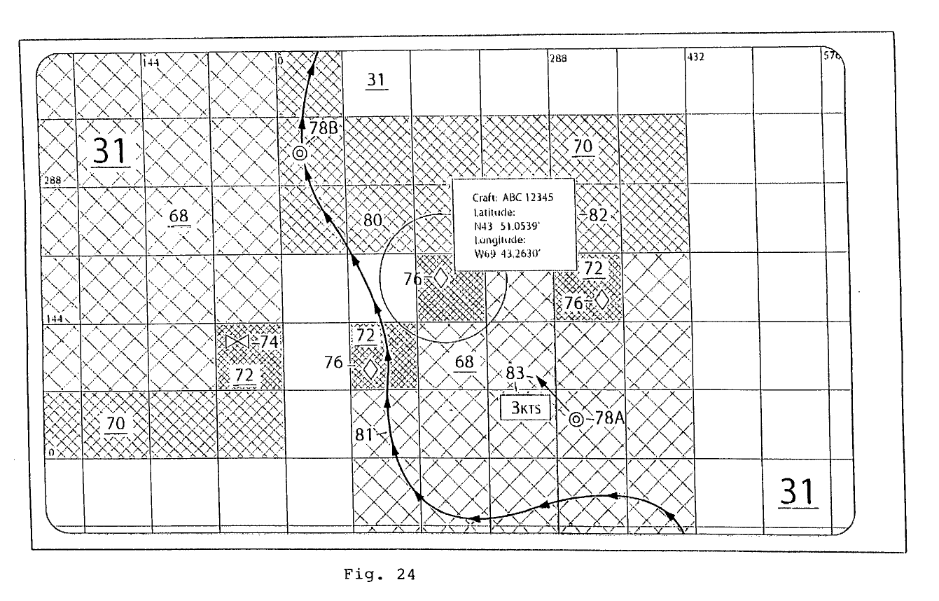

In FIG. 24 display view 4 shows a high level view of a coast guard search and rescue operation over water 31 with a search vessel 76 rescue path 81 that found initial reported point of interest 78A identified in an area already searched 68 and projected probable position of point of interest 78B in unsearched area along planned rescue path 81 based on prevailing current vector 83. A prevailing current flow beacon (not shown in FIG. 24) can be immediately dropped into the water 31, to increase the accuracy of prevailing current flows to improve the probability of the accuracy of predicted point of interest 78B. Improvement to the accuracy of the predicted point of interest 78B position can be achieved by having a first on arrival high speed low flying aircraft drop a string of current flow measuring beacon floats (or even an initial search grid of them) with Global Positioning System (GPS) transponder data to measure current flow to contribute to the accuracy of the predicted drift position in the display.

The known search areas on the water are very dynamic because of variance in ocean surface current that generally follows the prevailing wind, but with a series of drift beacons with the approximate dynamics as a floating person dropped along the original point of interest 78A (or as a grid), this drift flow prediction can be made much more accurate and allow the known and planned search areas to automatically adjust with the beacons in real-time. This can reduce the search time and improve the accuracy of predicted point of interest 78B, since unlike the land, the surface on the water moves with time and so would the known and unknown search areas.

An initial high speed rescue aircraft (or high speed jet drones) could automatically drop beacons at the intersections of a square grid (such as 1 mile per side, about a 100 beacons for 10 square miles) on an initial search, like along the grid lines of FIG. 24 where the search area would simply be warped in real-time with the position reports fed back from the beacons to re-shape the search grid in real time. Each flow measuring beacon can have a manual trigger switch and a flashing light so if a swimmer (that does not have a working Emergency Position Indicating Radio Beacon—EPIRB device) capable of swimming towards the beacon sees it and is able to get near it to identify they have been found. People are very hard to spot in the water even by airplane, and especially at night, and what makes it even more challenging is the currents move the people and the previously searched surfaces.

Another way to improve the search surface of FIG. 24 (and can be applied in other applications is use by border agents and by military to spot unfriendly's, friendly's, or intruders) can be by having a linear array of high powered infrared capable telescopic cameras (like an insect eye) mounted on a high speed aircraft zoomed (or telescoped) way-in, much farther than a human eye (like an eagle or birds eye, but having an array of them, such as 10, 20, or more telescopic views) and use high speed image processing for each telescopic camera to detect people. The current flow beacons as well as data automatically processed and collected by the telescopic sensor array can be used to augment the HUD360 1 see through display view 4.

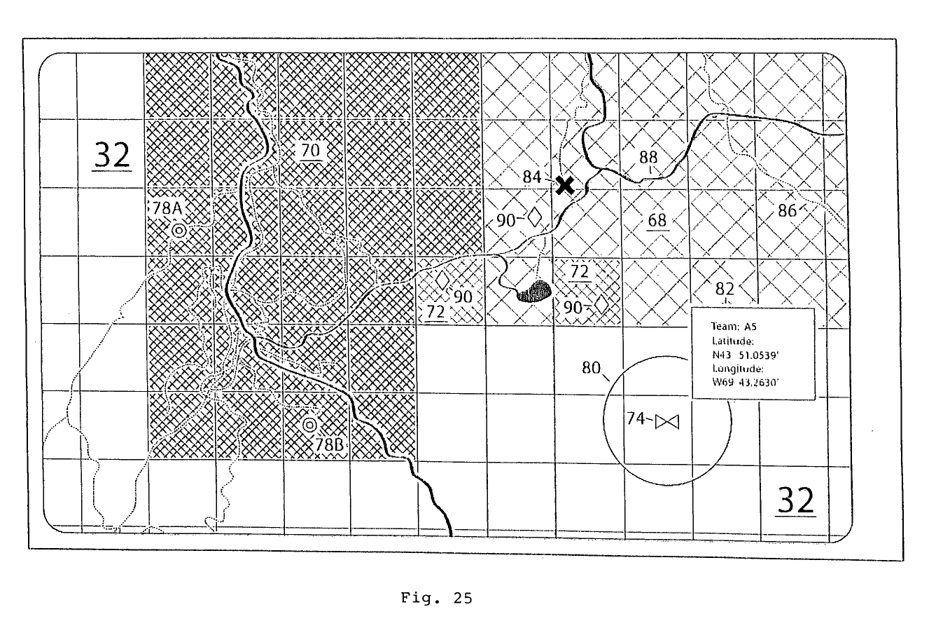

A ground search application view 4 of HUD360 1 is shown in FIG. 25 where a last known reported spotting of a hiker 84 was reported near ground search team positions 90 and rivers 88. The hikers reported starting position 78A and destination position 78B reported planned are shown along hiking trails 86. Search and rescue aircraft 74 is shown as selected search unit with selected data 82 shown. Although not shown in FIG. 25 the searched areas and searched hiking trails can be marked with appropriate colors to indicate if they have already searched and have the colors change as the search time progresses to indicate they may need to be searched again if the lost hiker has moved into that area based on how far nearby unsearched areas or trails are and a probable walking speed based on the terrain.

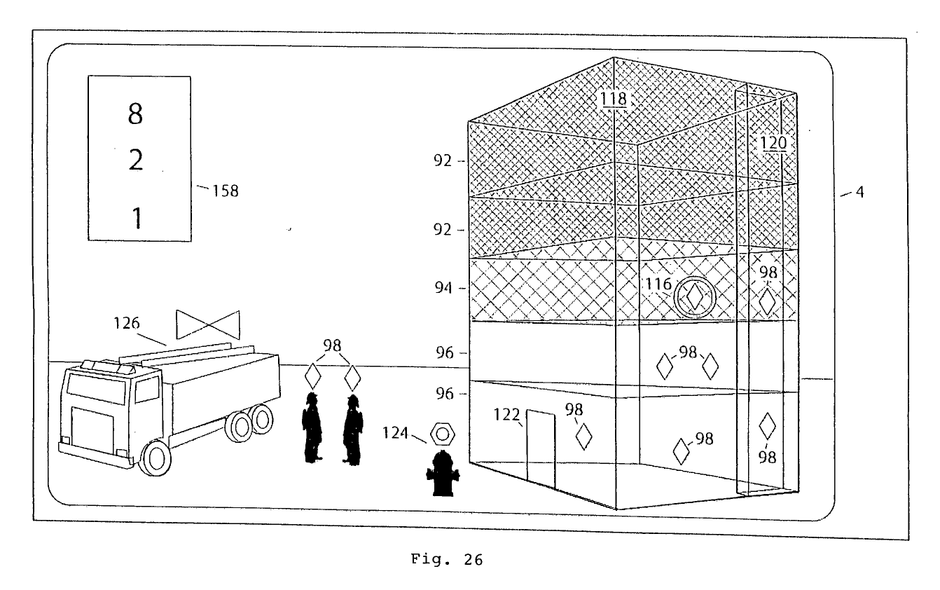

FIG. 26 shows an emergency response in see-through display view 4 to a building 118 under distress shown with stairwell 120, fire truck 126, fire hydrant 124, and main entrance 122. Inside the building 118 are floors in unknown state 92, floors actively being searched 94 and floors that are cleared 96. Firefighters 98 are shown outside and on the first three floors, with a distress beacon activated 116 on a firefighter on the third actively searched floor 94. Communications between HUD360 1 units can be achieved by using appropriate frequency bands and power levels that allow broadband wireless signals to propagate effectively and reliably through various building 118 structures, and repeaters can be added if necessary or the HUD360 1 itself can be used as a repeater to propagate broadband real-time data throughout the system. Broadcast data can also be sent to all HUD360 1 user's to order a simultaneous evacuation or retreat if sensors and building engineers indicate increasing probability of a building on the verge of collapsing or if some other urgency is identified, or just to share critical data in real-time.

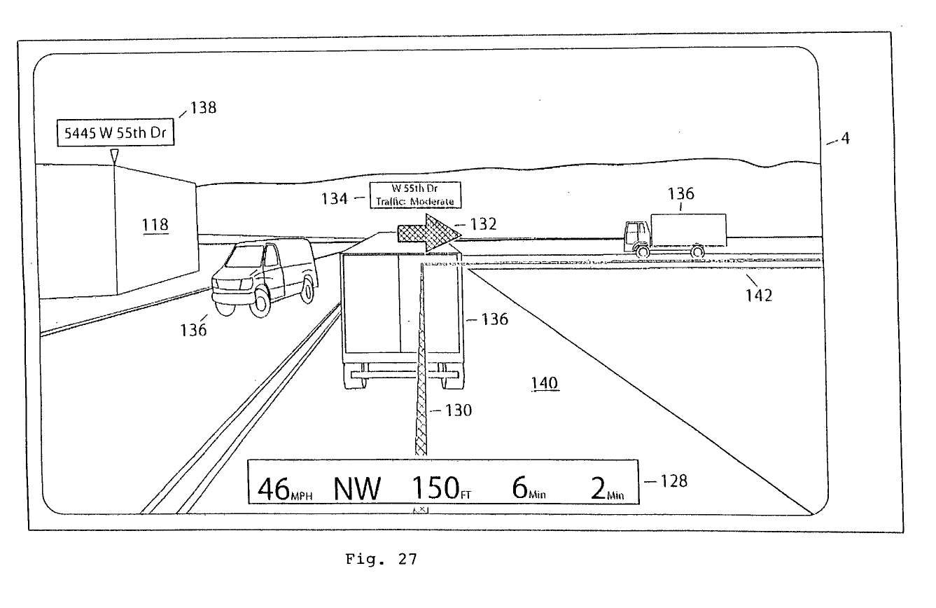

FIG. 27 shows a ground vehicle application view 4 of the HUD360 1 where a ground vehicle parameter display 128 is augmented onto the see-through display 4 on top of road 140 and planned route 130. Other vehicles 136 are shown on the road and can be augmented with data, such as speed and distance, as appropriate but not shown in FIG. 27. Upcoming turn indicator 132 is shown just below street and traffic status label 134 for road 142 to be turned on. Address label 138 is shown augmented on display 4 in the upper left of FIG. 27 used to aid the driver in identifying the addresses of buildings. The address label can be augmented to the corner of the building 118 by image processing such as segmentation of edges and known latitude and longitude of the building 118.

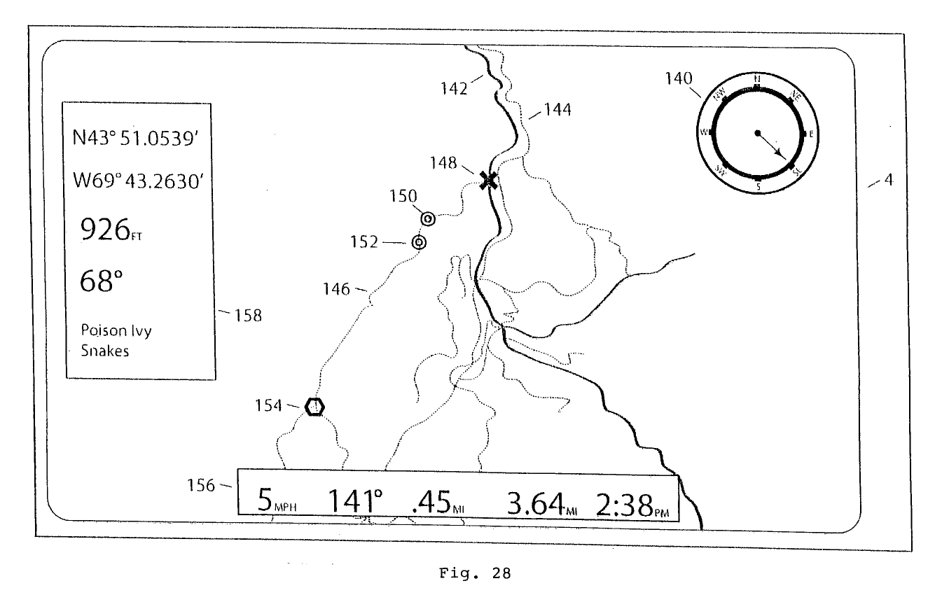

FIG. 28 shows a leisure hiking application view 4 of the HUD360 1 goggles in opaque mode with a map of the current hiking area with real time compass display 140, bottom parameter display 156 and side display 158 all of which can be augmented onto goggle display view 4 in see-through mode in addition to opaque mode shown in FIG. 28. Also shown in the display view 4 are rivers 142, inactive hiking trails 144 and active hiking trails 146. A destination cross-hair 148 is shown near the current position 150 with position of others in a group are shown as 152. A point of origin 154 is also shown near bottom left of trails 146 on display view 4. Various highlights of color not shown in FIG. 28 can be used to augment different real-time data or different aspects of the display view 4.

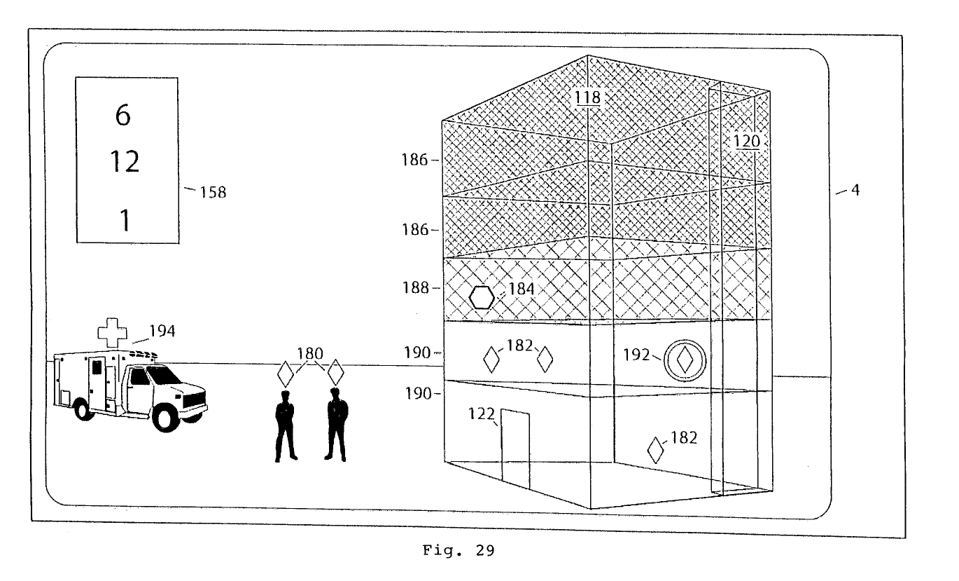

FIG. 29 shows a police or swat team application of a HUD360 1 see-through display view 4 with a side display augmentation 158 showing pertinent data relevant to the situation, with an emergency vehicle 194, police units on sight 180 with a building 118 in view. Inside the building police units not visible 182 are augmented on the first two floors marked as safe floors 190, where on the first floor a main entrance 122 is augmented. A second floor is shown augmented with an emergency beacon 192 as activated, and on the third floor is a probable hostage location 184 marked as the possible hostage floor 188. The top two floors (fifth and sixth) are marked as unknown floors 186, where the statuses of those floors are not currently known. Each personnel inside and outside the building or elsewhere can also be utilizing a HUD360 1 to assess the situation and better coordinate a rescue operation.

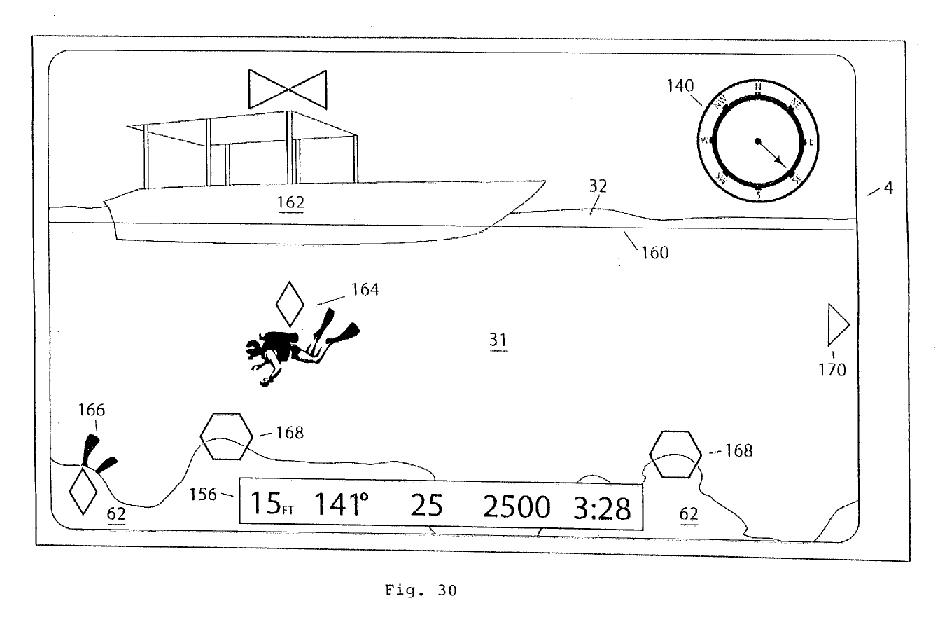

FIG. 30 shows a diver application augmented see-through display view 4 of a HUD360 1 with a dive boat 162 on top of water surface 160, in front of land 32, floating on top of water 31 shown with diver 164 below and diver 166 obstructed by reef 62 with high points 168 augmented. Also shown in FIG. 30 is an indicator of something of interest 170 on the right side of the see-through augmented display view 4 along with a parameter display 156 at bottom of augmented see-through display view 4 with critical dive parameters to aid the diver in having a safer diving experience.

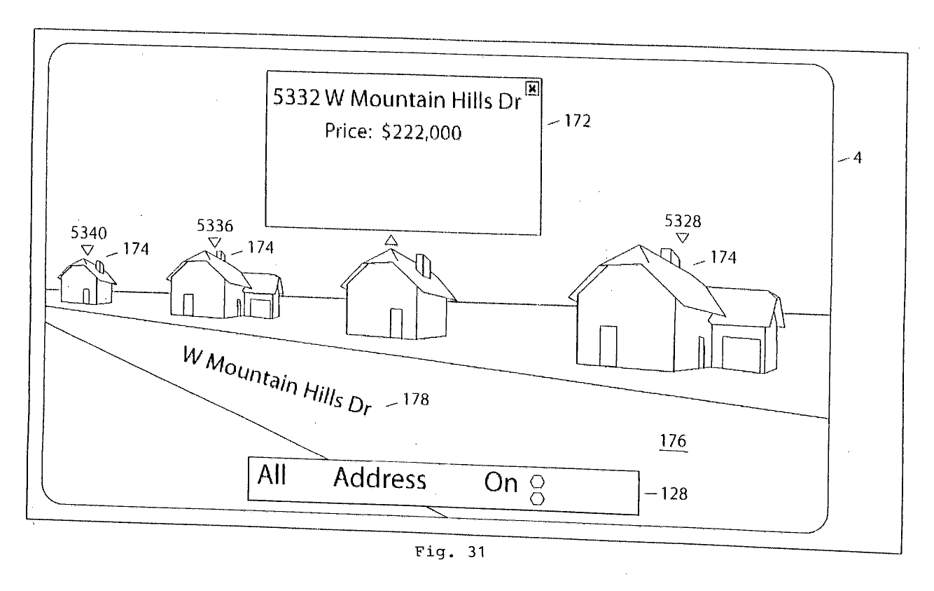

FIG. 31 shows a HUD360 1 application see-through display view 4 for a real estate agent providing augmented display data on a selected house 172 showing any details desired, including a virtual tour, among other homes not selected 174 along street 176 with street label 178, and vehicle data display 128 augmented with real estate data on bottom of see-through display view 4 shown. Address labels are augmented on the see-through display view 4 above selected homes 174 using latitude and longitude data along with head-orientation data to align the address labels above the homes.

All the FIGS. Shown herein show different display modes that are interchangeable for each application, and is meant to be just a partial example of how augmentation can be displayed. The applications are not limited to one display mode. For instance, FIG. 31 shows a ground view, but can also show a high level opaque mode view of the property a view high above ground looking down.

This invention is not limited to aircraft, but can be just as easily applied to automobiles, ships, aircraft carriers, trains, spacecraft, or other vessels, as well as be applied for use by technicians or mechanics working on systems.
1. An ATC system for automatically receiving tactical and environmental data from multiple aircraft positions and displaying 3 dimensional aircraft data, displaying 3 dimensional weather data, displaying 3 dimensional terrain, and 3 dimensional ground obstacles by transforming these images into a 3 dimensional orthogonal space on the COTS light weight projection glasses that allows the user to:
   a. Perfectly line up the projected image directly overlaying the real aircraft, terrain, and obstacle objects.
   b. Select an object on the display and presenting known information about the object from an accompanying database.
   c. View the moving objects current attributes, such as velocity, direction, altitude, vertical speed, projected path, etc.
   d. View the terrain and obstacle object's attributes, such as latitude, longitude, elevation, etc.
   e. View all moving aircraft flight plans, if the aircraft has a Flight Management flight plan and Automatic Dependent Surveillance Broadcast (ADS-B) or other comparable data link functionality.
   f. Track each objects predicted position vector and flight plan, if available, to determine if a collision is anticipated, either in the air or on the ground taxiway, and provide a warning when an incursion is projected.
   g. View the tactical situation from the point of view of a selected object allowing ATC to view the traffic from a pilot's point of view.
   h. View ground traffic, such as taxiing aircraft.
   i. Display ground obstacles in 3D from data in an obstacle database.
   j. Update the 3 dimensional augmentations on the COTS light weight projection glasses based on movement of the user's head.
   k. Allow selection and manipulation of 3 dimensional augmentations or other augmentation display data by combining eye tracking and head tracking with or without voice command and/or button activation.
   l. Identify and augment real-time space type categorization.
2. A pilot cockpit system for automatically receiving tactical and environmental data from multiple aircraft positions, its own aircraft position and displaying 3 dimensional aircraft data, displaying 3 dimensional weather data, displaying 3 dimensional terrain, and 3 dimensional ground obstacles by transforming these images into a 3 dimensional orthogonal space on the COTS light weight projection glasses that allows the user to:
   a. Perfectly line up the projected image directly overlaying the real aircraft, terrain, and obstacle objects.
   b. Select an object on the display and presenting known information about the object from an accompanying database.
   c. View the moving objects current attributes, such as velocity, direction, altitude, vertical speed, projected path, etc.
   d. View the terrain and obstacle objects attributes, such as latitude, longitude, elevation, etc.
   e. View own aircraft flight plan, if the object has a Flight Management flight plan and ADS-B capability or other comparable data link functionality.
   f. View other aircraft flight plan, if the object is an aircraft and has ADS-B capability or other comparable data link functionality enabled.
   g. Track each objects predicted position vector and flight plan, if available, to determine if a collision is anticipated, either in the air or on the ground taxiway, and provide a warning when an incursion is projected.
   h. View ground traffic, such as taxiing aircraft.
   i. Update the 3 dimensional augmentations on the COTS light weight projection glasses based on movement of the user's head.
   j. Allow selection and manipulation of 3 dimensional augmentations or other augmentation display data by combining eye tracking and head tracking with voice command and/or button activation.
   k. Identify and augment real-time space type categorization.
3. A military battlefield system for automatically receiving tactical and environmental data from aircraft, tanks, ground troops, naval ships, painted enemy positions, etc. and displaying 3 dimensional battlefield objects, displaying 3 dimensional weather data, displaying 3 dimensional terrain, and 3 dimensional ground obstacles by transforming these images into a 3 dimensional orthogonal space on the COTS light weight projection glasses that allows the user to:
   a. Perfectly line up the projected image directly overlaying the real object.
   b. Select an object on the display and presenting known information about the object.
   c. View the objects current attributes, such as relative distance, velocity, direction, altitude, vertical speed, projected path, etc.
   d. View enemy objects.
   e. View Joint STARS data.
   f. Track each objects predicted position vector and identify battlefield conflicts and spaces.
   g. View the tactical situation from the point of view of a selected object to allow the user to see a battlefield from any point of the battlefield.
   h. See where friendly troops are to gain a tactical advantage on a battlefield.
   i. Update the 3 dimensional augmentations on the COTS light weight projection glasses based on movement of the user's head.
   j. Allow selection and manipulation of 3 dimensional augmentations or other augmentation display data by combining eye tracking and head tracking with voice command and/or button activation.
   k. Identify and augment real-time space type categorization.
4. An automotive system for automatically receiving tactical and environmental data from the current automobile position, traffic advisories, etc., and displaying 3 dimensional weather data, displaying 3 dimensional terrain, and 3 dimensional ground obstacles by transforming these images into a 3 dimensional orthogonal space on the COTS light weight projection glasses that allows the user to:
   a. Perfectly line up the projected image directly overlaying the real object.
   b. Select an object on the display and presenting known information about the object from an accompanying database.
   c. View traffic advisory information.
   d. View current weather conditions.
   e. View current route.
   f. Allow the user to modify the route through voice commands.
   g. Identify and augment real-time space type categorization.
5. A medical system viewing the inside of a patient from non-invasive patient data such as MRI, CAT scan, etc. or by using a surgical probe to allow doctors to view the internal organs, tumors, broken bones, etc. by transforming these images into a 3 dimensional orthogonal space on the COTS light weight projection glasses that allows the user to:
   a. Rotate the patient's image to view the patient from the inside.
   b. Identify tumors, cancerous areas, etc before operating on the patient.
   c. Allow the doctor to practice the procedure before operating on the patient.
   d. Allow doctors to look at different ways to do an operation without putting the patient in peril.
   e. Allow new doctors to practice and develop surgical skills without operating on a live patient.
   f. Allow doctors to view the inside of the body in 3 dimensions using Arthroscopic camera technology.
   g. Allow vision impaired people to read as well as watch television and movies.
   h. Identify and augment real-time space type categorization.
6. A system, according to claims 1 through 5, characterized by further enabling the user to use voice commands to select any options allowed by the system.
7. A system, according to claims 1 through 5, characterized by further enabling training by using a set of user definable objects in the controlled area.
8. A system, according to claims 1 through 5, characterized by allowing the recording of the controlled area over a specified time period to allow later playback of all actions in the controlled area.

We claim:

1. A process for medical imaging comprising the steps of
a. providing non-invasive patient data derived from technology selected from the group comprising magnetic resonance imagery, X-ray, CAT scan, and sonogram;
b. providing COTS lightweight projection glasses;
c. transforming said patient data into images; and
d. transforming said images into three dimensional orthogonal space on said COTS lightweight projection glasses.

2. A process for medical imaging comprising the steps of
a. providing a surgical probe with imaging capabilities;
b. inserting said surgical probe into a patient;
c. collecting patient data with said imaging capabilities;
d. providing COTS lightweight projection glasses;
e. transforming said patient data into images; and
f. transforming said images into three dimensional orthogonal space on said COTS lightweight projection glasses.

3. A process of navigating in three dimensional space comprising the steps of
a. providing a database;
b. providing at least one sensor;
c. providing a controller;
d. providing an augmented reality display means;
e. providing networking means connecting said database, said at least one sensor, said controller, and said augmented reality display means; and
f. presenting data from said database, said at least one sensor, said controller, and said augmented reality display means to a user.

4. The process of claim 3 wherein said user operates a vehicle; said database, said at least one sensor, and said controller are aboard said vehicle; said user wears said augmented reality display means; and said augmented reality display means presents an augmented see through display.

5. The process of claim 3 wherein said user operates a vehicle; said networking means has broadband communication means; said augmented reality display means presents an augmented see through display; and said controller uses data from said sensor continually to update said database and said augmented see through display.

6. The process of claim 3 wherein said networking means has broadband communication means and can communicate with a plurality of remote stations; said augmented reality display means presents an augmented see through display; said controller can assess data from said database and said sensor to determine attributes of objects in said three dimensional space; said attributes are selected from the group comprising threat, distance, velocity, size, position, price, address, depth, heading, time, identity, and resource availability; and said controller projects said attributes onto said augmented reality display means.

7. The process of claim 3 wherein said augmented reality display means presents an augmented see through display; said controller uses data from said sensor continually to update said database and said augmented see through display; said controller can assess data from said database and said sensor to determine attributes of objects in said three dimensional space; said attributes are selected from the group comprising threat, distance, velocity, size, position, price, address, depth, heading, time, identity, and resource availability; and said controller projects said attributes onto said augmented reality display means.

8. The process of claim 7 further comprising the step of providing a user input means; said user operates a vehicle; and said attributes are displayed to said user in response to signals from said user input means.

9. The process of claim 8 wherein said attributes are displayed so that said user can perceive said objects in real time in three dimensional space even if line of sight to said objects in three dimensional space is occluded; and said user input means comprises data obtained through said sensor and selected from the group comprising eye orientation, head orientation, voice command, and push button.

10. The process of claim 7 wherein said attributes are displayed so that said user can perceive said objects in three dimensional space even if line of sight to said objects in three dimensional space is occluded.

11. The process of claim 3 wherein said at least one sensor comprises a plurality of sensors selected from the group comprising radar, orientation sensors, visible spectrum cameras, infrared cameras, microphones, transceivers, clocks, thumb position sensors, computer mouses, pointing sensors, global positioning system transponders, MRI, CAT scan, fuel sensors, speedometer, thermometer, depth sensor, pressure sensor, X-ray, sonogram, and wind sensors.

12. The process of claim 3 wherein said at least one sensor comprises a plurality of sensors mounted on a platform selected from the group comprising a satellite, a vehicle operated by said user, a beacon, an air traffic control tower, a military control center, a display means worn by said user, and a vehicle not operated by said user.

13. The process of claim 3 wherein said database contains data that can be updated by means selected from the group comprising said at least one sensor, known data sources, neural network, fuzzy logic, time based decaying weights, assigned paths, official chart data, and plans.
